(19) United States
(12) Patent Application Publication
(10) Pub. No.: US 2019/0135911 A1
(43) Pub. Date: May 9, 2019

(54) TREATING RESPIRATORY DISEASES BY TARGETING INTERLEUKIN 4 INDUCED 1 (IL4I1)

(71) Applicants: Five Prime Therapeutics, Inc., South San Francisco, CA (US); GLAXOSMITHKLINE INTELLECTUAL PROPERTY DEVELOPMENT LIMITED, Brentford, Middlesex (GB)

(72) Inventors: Kathleen M. SULLIVAN, San Mateo, CA (US); Katherine Kurylo, South San Francisco, CA (US); Paul Redford, Stevenage (GB); Soren Beinke, Stevenage (GB); David Michalovich, Stevenage (GB); Karen Simpson, Stevenage (GB); Edith Hessel, Stevenage (GB)

(21) Appl. No.: 16/096,699

(22) PCT Filed: Apr. 21, 2017
(86) PCT No.: PCT/US2017/028806

§ 371 Date: Oct. 26, 2018

Related U.S. Application Data
(60) Provisional application No. 62/327,718, filed on Apr. 26, 2016.

(51) Int. Cl. C07K 16/24; A61P 11/00; A61P 31/22; A61P 31/06

(52) U.S. Cl. CPC C07K 16/247; A61P 31/06; A61P 31/22; A61P 11/00

(57) ABSTRACT

Provided herein are methods for treating or preventing a respiratory disease, such as chronic obstructive pulmonary disease (COPD), by reducing or inhibiting interleukin 4 inducible 1 (IL4I1) activity.

### CROSS-REFERENCE TO RELATED APPLICATION

This application claims priority to U.S. Provisional Application No. 62/327,718 filed Apr. 26, 2016, herein incorporated by reference.

### FIELD

This application relates to methods of treating or preventing a respiratory disease, such as chronic obstructive pulmonary disease (COPD), by reducing or inhibiting interleukin 4 induced 1 (IL4I1) activity, for example by use of an antagonist specific for IL4I1 protein or downstream products or targets thereof.

### PARTIES TO JOINT RESEARCH AGREEMENT

Five Prime Therapeutics, Inc. and GlaxoSmithKline are parties to a joint research agreement governing inventions disclosed herein.

### BACKGROUND

Interleukin 4 induced 1 (IL4I1), also known as FIG. 1, was originally identified in B cells following treatment with IL4. IL4I1 is a lysosomal protein, which can also be expressed and secreted by dendritic cells. IL4I1 has similarity with L-amino acid oxidase, and has high specificity for the substrate phenylalanine. Two alternatively spliced transcript variants of this gene encoding two distinct isoforms have been reported.

Reported activities for IL4I1 protein include lysosomal antigen processing and presentation, suppression of Th1, Th2 and Th17 responses, and promoting a M2 macrophage phenotype.

### SUMMARY

The data herein demonstrate the role of IL4I1 in respiratory disease, such as COPD. Based on these observations, provided are methods of treating or preventing a respiratory disease in a subject including administering to the subject a therapeutically effective amount of one or more antagonists that reduce or inhibit IL4I1 activity (e.g., an antagonist specific for an IL4I1 protein or downstream products or targets thereof), such as an antagonist that alters signalling, expression, or activity of IL4I1 (such as inhibitory molecules specific for IL4I1), or an antagonist that alters signalling, expression, or activity of at least one downstream target of IL4I1. Thus, also provided are uses for one or more antagonists that reduce or inhibit IL4I1 activity in the manufacture of a medicament for the treatment or prevention of a respiratory disease. Also provided are antagonists that reduce or inhibit IL4I1 activity for use in the treatment or prevention of a respiratory disease. Also provided are pharmaceutical compositions for use in the treatment or prevention of a respiratory disease, wherein the composition includes one or more antagonists that reduce or inhibit IL4I1 activity, together with at least one pharmaceutical carrier, diluent or excipient.

Exemplary respiratory diseases that can be treated or prevented with the disclosed methods and compositions include, but are not limited to: COPD, exacerbated COPD, asthma, bronchitis, emphysema, cystic fibrosis, acute respiratory distress syndrome, bronchiectasis, or combinations thereof. In some examples, the COPD exacerbation results from a respiratory infection, such as a bacterial infection (e.g., infection by one or more of Moraxella catarrhalis, Haemophilus influenzae, Streptococcus pneumoniae and/or mycobacteria such as Mycobacterium tuberculosis) or a viral infection (e.g., infection by human rhinovirus (HRV) or Respiratory syncytial virus (RSV)). In some examples, the respiratory infection is a secondary bacterial infection. Thus, subject can have COPD, exacerbated COPD, asthma, bronchitis, emphysema, cystic fibrosis, acute respiratory distress syndrome, bronchiectasis, or combinations thereof. In some examples, the subject has a respiratory infection, such as due to a bacterial infection (e.g., infection by one or more of Moraxella catarrhalis, Haemophilus influenzae, Streptococcus pneumoniae, and/or mycobacteria such as Mycobacterium tuberculosis) or a viral infection (e.g., infection by HRV or RSV).

In one embodiment, the IL4I1 activity is reduced or inhibited by one or more antagonists of an IL4I1 protein and/or nucleic acid molecule. In another embodiment, the antagonist of an IL4I1 nucleic acid molecule comprises an inhibitory nucleic acid molecule, such as an antisense nucleic acid molecule, small interfering RNA (siRNA), short hairpin RNA (shRNA), micro RNA (miRNA), ribozyme, or combination thereof, specific for an IL4I1 nucleic acid molecule. In one example, the antagonist of an IL4I1 protein comprises an antibody, antibody fragment, antibody conjugate, domain antibody” or “dAb™, small organic molecule, small inorganic molecule, or combination thereof, specific for an IL4I1 protein. In some examples, the an IL4I1 activity is reduced or inhibited by one or more antagonists of a downstream target of IL4I1.

Also provided are methods of screening for an antagonist of IL4I1 activity for use in treating or preventing a respiratory disease.

The foregoing and other objects and features of the disclosure will become more apparent from the following detailed description, which proceeds with reference to the accompanying figures.

### BRIEF DESCRIPTION OF THE DRAWINGS

FIG. 1 includes a series of charts showing a selection of differentially expressed genes (CXCL5, PTGES, S100A8, SERPINB2, and THBS1) induced in healthy monocyte derived DC (moDC) following co-culture with COPD bronchial epithelial cells (BEC), as compared to healthy BEC.

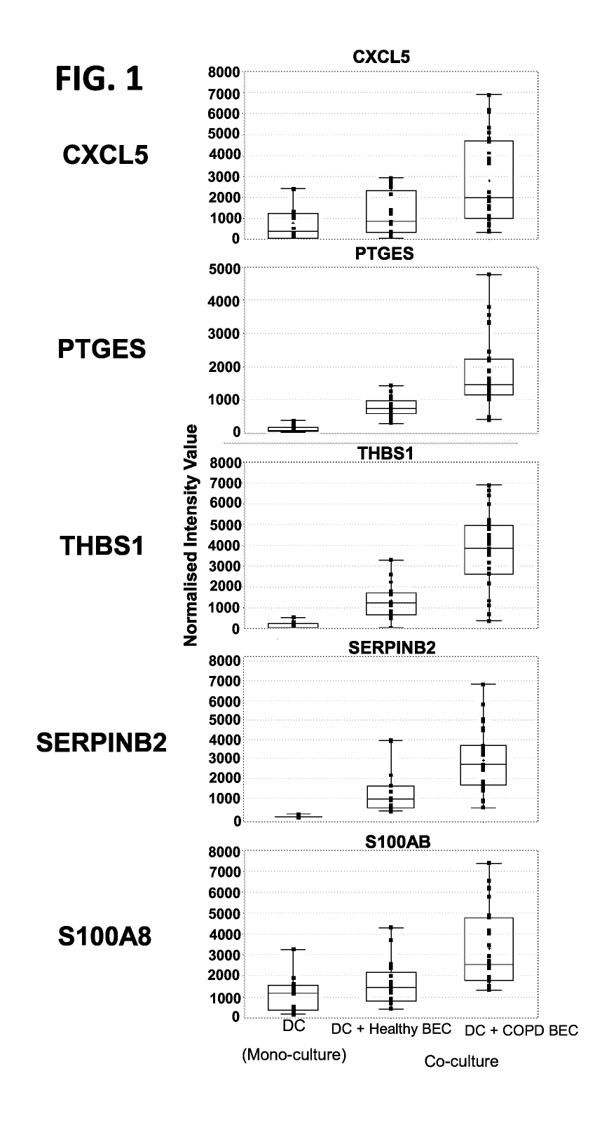

FIG. 2 includes a series of charts showing that treatment of moDC in co-culture with epithelial cells from COPD patients with IL4I1 results in the enhanced mRNA expression of dendritic cell genes such as CXCL5, PTGES, S100A8, SERPINB2, and THBS1.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an antagonist, such as one or more antagonists that reduce or inhibit IL4I1 activity (e.g., an antagonist specific for one or more IL4I1 nucleic acid molecules or proteins or downstream products or targets thereof), by any effective route. Exemplary routes of administration include, but are not limited to, oral, injection (such as subcutaneous, intramuscular, intradermal, intraperitoneal, intravenous, and intratumoral), sublingual, rectal, transdermal, intranasal, vaginal and inhalation routes.

Antibody: A polypeptide including at least a light chain or heavy chain immunoglobulin variable region which specifically recognizes and binds an epitope of an antigen, such as IL4I1, or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for IL4I1 and in some examples also reduce or inhibit the biological activity of an IL4I1 protein.

The term antibody includes intact immunoglobulins, as well the variants and portions thereof, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target protein will have a specific V_{H} region and the V_{L} region sequence, and thus specific CDR sequences. Antibodies with different specificities (such as different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species, such as human, and CDRs (which generally confer antigen binding) from another species, such as a murine antibody that specifically binds IL4I1.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one example, all the CDRs are from the donor immunoglobulin in a humanized immunoglobulin. Constant regions need not be present, but if they are, they are substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized immunoglobulins can be constructed by means of genetic engineering (see for example, U.S. Pat. No. 5,585,089).

Single variable domain: The term “single variable domain” refers to a folded polypeptide domain comprising sequences characteristic of antibody variable domains. It therefore includes complete antibody variable domains such as VH, VHH and VL and modified antibody variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain. A single variable domain is capable of binding an antigen or epitope independently of a different variable region or domain. A “domain antibody” or “dAb™” may be considered the same as a “single variable domain”. A single variable domain may be a human single variable domain, but also includes single variable domains from other species such as a rodent or Camelid VHH dAbs™. Camelid VHH are immunoglobulin single variable domain polypeptides that are derived from species including camel, llama, alpaca, dromedary, and guanaco, which produce heavy chain antibodies naturally devoid of light chains. Such VHH domains may be humanised according to standard techniques available in the art, and such domains are considered to be “single variable domains”. As used herein VH includes camelid VHH domains.

Binding: An association between two substances or molecules, such as the hybridization of one nucleic acid molecule to another (or itself), the association of an antibody, or functional nucleic acid (such as an aptamer) with a protein or small organic molecule, or the association of a protein with another protein or nucleic acid molecule. Binding can be detected by any procedure known to one skilled in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorptionlionization time-of-flight mass spectrometry, microcytometry, microarray, microscopy, fluorescence activated cell sorting (FACS), and flow cytometry.

One molecule is said to “specifically bind” to another molecule when a particular antagonist (a “specific binding antagonist”) can specifically react with a particular target, but not to unrelated molecules, for example to specifically immunoreact with a target, to specifically hybridize to a target, or to specifically bind to a target. For example, an IL4I1 specific binding antagonist binds substantially only to the IL4I1 protein in vitro or in vivo. The binding is a non-random binding reaction, for example between a specific binding antagonist (such as an antibody or functional fragment thereof, protein, nucleic acid molecule or functional nucleic acid molecule) and a target (such as a cell, protein, DNA or RNA). Binding specificity can be determined from the reference point of the ability of the specific binding antagonist to differentially bind the target and an unrelated molecule, and therefore distinguish between two different molecules. For example, an oligonucleotide molecule binds or stably binds to a target nucleic acid molecule if a sufficient amount of the oligonucleotide molecule forms base pairs or is hybridized to its target nucleic acid molecule, to permit detection of that binding.

In some examples, a molecule (such as an antibody) specifically binds to a target (such as a protein) with a binding constant that is at least 10³ M⁻¹ greater, 10⁴M⁻¹ greater or 10⁵ M⁻¹ greater than a binding constant for other molecules in a sample or subject. In particular examples, two compounds are said to specifically bind when the binding constant for complex formation between the components is at least 10⁴ L/mol, for example, at least 10⁶ L/mol, at least 10⁸ L/mol, or at least 10¹⁰ L/mol. The binding constant for two components can be determined using methods that are well known in the art.

Aptamers: Aptamers are nucleic acid molecules having specific binding affinity to molecules through interactions other than classic Watson-Crick base pairing. Aptamers, like peptides generated by phage display or monoclonal antibodies (MAbs), are capable of specifically binding to selected targets and, through binding, block their targets' ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., will typically not bind other proteins from the same gene family) A series of structural studies have shown that aptamers are capable of using the same types of binding interactions (hydrogen bonding, electrostatic complementarity, hydrophobic contacts, steric exclusion, etc.) that drive affinity and specificity in antibody-antigen complexes.

Aptamers have a number of desirable characteristics for use as therapeutics including high specificity and affinity, biological efficacy, and excellent pharmacokinetic properties. In addition, they offer specific competitive advantages over antibodies and other protein biologics, for example:
1) Speed and control. Aptamers are produced by an entirely in vitro process, allowing for the rapid generation of initial therapeutic leads. In vitro selection allows the specificity and affinity of the aptamer to be tightly controlled and allows the generation of leads against both toxic and non-immunogenic targets.
2) Toxicity and Immunogenicity. Aptamers as a class have demonstrated little or no toxicity or immunogenicity. In chronic dosing of rats or woodchucks with high levels of aptamer (10 mg/kg daily for 90 days), no toxicity is observed by any clinical, cellular, or biochemical measure. Whereas the efficacy of many monoclonal antibodies can be severely limited by immune response to antibodies themselves, it is extremely difficult to elicit antibodies to aptamers (most likely because aptamers cannot be presented by T-cells via the MHC and the immune response is generally trained not to recognize nucleic acid fragments).
3) Administration. Whereas all currently approved antibody therapeutics are administered by intravenous infusion (typically over 2-4 hours), aptamers can be administered by subcutaneous injection. This difference is primarily due to the comparatively low solubility and thus large volumes are necessary for most therapeutic MAbs. With good solubility (>150 mg/ml) and comparatively low molecular weight (aptamer: 10-50 KD; antibody: 150 KD), a weekly dose of aptamer may be delivered by injection in a volume of less than 0.5 ml. In addition, the small size of aptamers allows them to penetrate into areas of conformational constrictions that do not allow for antibodies or antibody fragments to penetrate, presenting yet another advantage of aptamer-based therapeutics or prophylaxis.
4) Scalability and cost. Therapeutic aptamers are chemically synthesized and consequently can be readily scaled as needed to meet production demand Whereas difficulties in scaling production are currently limiting the availability of some biologics and the capital cost of a large-scale protein production plant is enormous, a single large-scale synthesizer can produce upwards of 100 kg oligonucleotide per year and requires a relatively modest initial investment. The current cost of goods for aptamer synthesis at the kilogram scale is estimated at S500/g, comparable to that for highly optimized antibodies. Continuing improvements in process development are expected to lower the cost of goods to <100/g in five years.
5) Stability. Therapeutic aptamers are chemically robust. They are intrinsically adapted to regain activity following exposure to heat, denaturants, etc. and can be stored for extended periods (>1 yr) at room temperature as lyophilized powders. In contrast, antibodies must be stored refrigerated.

Chronic obstructive pulmonary disease (COPD): A type of obstructive lung disease characterized by chronically poor airflow, which typically worsens over time. Exemplary symptoms include shortness of breath, cough, and sputum production. Some people with chronic bronchitis have COPD. Causes of COPD can include smoking, air pollution, and genetics. Exacerbation of COPD, a sudden, worsening, or flare-up, of baseline COPD symptoms, can be evidenced by an increased shortness of breath (dyspnea), increased sputum production, a change in the color of the sputum from clear to green or yellow, and/or an increase in cough in someone with COPD. This may present with signs of increased work of breathing such as fast breathing, a fast heart rate, sweating, active use of muscles in the neck, a bluish tinge to the skin, and confusion or combative behavior in very severe exacerbations. Crackles may also be heard over the lungs on examination with a stethoscope. In some cases, acute exacerbation is triggered by infection (such as bacteria, virus, or both), environmental pollutants, or improper use of medications.

IL4I1: IL4I1 expression can be induced in specific cell types (for example in B cells, but not T lymphocytes nor mast cells), following treatment with IL4. IL4I1 contains several residues important for substrate binding and catalysis. IL4I1 includes an N-terminal signal peptide, a central region that shares homology with nonmammalian L-amino acid oxidases, and 3 conserved domains that may be involved in FAD binding. IL4I1 has a preference for aromatic amino acid substrates, particularly phenylalanine. IL4I1 is a secreted, N-glycosylated enzyme located in germinal center macrophages and inflammatory myeloid cells. IL4I1 co-localizes with lysosomal dyes, indicating higher enzymatic activity at acidic pH, and a possible role in lysosomal antigen processing and presentation. There is also evidence that IL4I1 is associated with suppression of Th1, Th2 and Th17 responses, and promoting a M2 macrophage phenotype.

IL4I1 activity: The term “IL4I1 activity” refers to the biological activity, which may be reduced or inhibited by use of an antagonist of IL4I1, for example, by reducing the catalytic activity of IL4I1 and/or preventing signaling from IL4I1 and/or downstream targets.

Isolated: An “isolated” biological component (such as an IL4I1 protein, antibody, or nucleic acid molecule) has been substantially separated, produced apart from, or purified away from other biological components in the cell of the organism in which the component occurs, such as other chromosomal and extrachromosomal DNA and RNA, and proteins. Nucleic acids molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, antibody, protein, or nucleic acid molecule can be at least 70%, at least 80%, at least 90%, at least 95%, at least 96%, at least 97%, at least 98%, or at least 99% pure.

Mammal: This term includes both human and non-human mammals. Similarly, the term “subject” includes both human and veterinary subjects (such as cats, dogs, horses, cows, and pigs) and rodents (such as mice and rats).

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the antagonists provided herein such as one or more antagonists that reduce or inhibit Il4i1 activity (e.g., an antagonist specific for a IL4I1 nucleic acid molecule or protein or downstream product or target thereof).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying antagonists, preservatives, and pH buffering agents and the like, for example sodium acetate or sorbitan monolaurate.

Purified: The term purified does not require absolute purity; rather, it is intended as a relative term. Thus, for example, a purified peptide or antibody preparation is one in which the peptide or protein is more enriched than the peptide or protein is in its natural environment within a cell. In one embodiment, a preparation is purified such that the protein or antibody represents at least 50%, 60%, 70%, 80%, 90%, 95%, 96%, 97%, 98%, or 99% of the total antibody or protein content of the preparation.

Respiratory disease: A disease associated with a pathological condition of the upper respiratory tract, bronchi, bronchioles, alveoli, pleura, and/or pleural cavity. In some examples, a respiratory disease is an inflammatory lung disease, such as one characterized by an elevated neutrophil count (e.g., asthma, emphysema, COPD, cystic fibrosis, and acute respiratory distress syndrome). In some examples, a respiratory disease is caused by an infection, such as an upper or lower respiratory tract infection, such as one due to a viral or bacterial infection. In one example, the respiratory disease is not due to a cancer or tumor of the lung. Examples of respiratory diseases that can be treated or prevented by the disclosed methods include one or more of COPD, exacerbated COPD, asthma, bronchitis, emphysema, cystic fibrosis, acute respiratory distress syndrome, bronchiectasis and the like.

Small molecule: A molecule, typically with a molecular weight less than about 1000 Daltons, or in some embodiments, less than about 500 Daltons, wherein the molecule is capable of modulating, to some measurable extent, an activity of a target molecule, such as IL4I1.

Specific binding antagonists: An antagonist that binds substantially or preferentially only to a defined target, such as a target protein or nucleic acid molecule. In an example, a “specific binding antagonist” is capable of binding to IL4I1. In other examples, the specific binding antagonist is capable of binding to a downstream target of IL4I1 or factor regulated by IL4I1. A protein-specific binding antagonist binds substantially only the target protein, or to a specific region within the protein. For example, a “specific binding antagonist” includes antibodies, antibody fragments, and other antagonists that bind substantially to a specified polypeptide, such as small molecules, aptamers or other functional nucleic acid molecules. The determination that a particular antagonist binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999).

Therapeutically effective amount: An amount of one or more antagonists that reduce or inhibit IL4I1 activity (e.g., an antagonist specific for an IL4I1 nucleic acid molecule or protein or downstream product or target thereof), that alone, or together with an additional therapeutic antagonist (s), is sufficient to prevent, treat (including prophylaxis), reduce and/or ameliorate the symptoms and/or underlying causes of any of a disorder or disease. In one embodiment, an “effective amount” is sufficient to reduce or eliminate a symptom of a disease, such as a respiratory disease (such as COPD or exacerbated COPD), for example by reducing neutrophil count, reducing sputum production, reducing shortness of breath, reducing cough, improving pulmonary function, or combinations thereof.

A therapeutically effective amount of one or more antagonists that reduce or inhibit IL4I1 activity (e.g., an antagonist specific for an IL4I1 nucleic acid molecule or protein or downstream product or target thereof) can be administered in a single dose, or in several doses, for example daily, during a course of treatment. However, the therapeutically effective amount can depend on the subject being treated, the severity and type of the condition being treated, the manner of administration and the type of therapeutic antagonist being administered.

Tissue: A plurality of functionally related cells. A tissue can be a suspension, a semi-solid, or solid. Tissue includes cells collected from a subject, such as the lung.

Treating a disease: “Treatment” refers to a therapeutic intervention that ameliorates a sign or symptom of a disease or pathological condition after it has begun to develop, such a sign or symptom of a respiratory disease (such as COPD or exacerbated COPD). Treatment can also induce remission or cure of a condition, such as a respiratory disease. Preventing a disease refers to a therapeutic intervention to a subject who does not exhibit signs of a disease or exhibits only early signs for the purpose of decreasing the risk of developing pathology, such that the therapy inhibits or delays the full development of a disease, such as preventing development of a respiratory disease (such as COPD or exacerbated COPD). Treatment and prevention of a disease does not require a total absence of disease. For example, a decrease of at least 20% or at least 50% can be sufficient. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

Under conditions sufficient for: A phrase that is used to describe any environment that permits the desired activity. In one example, includes administering a therapeutic antagonist to a cell or a subject sufficient to allow the desired activity. In particular examples, the desired activity is decreasing the activity of IL4I1.

### Methods of Treating or Preventing a Respiratory Disease

Provided are methods of treating or preventing a respiratory disease in a subject. Such methods can include administering to a subject a therapeutically effective amount of one or more antagonists that reduce or inhibit IL4I1 activity (e.g., an antagonist specific for an IL4I1 nucleic acid molecule or protein or downstream product or target thereof), such as an antagonist that alters expression or activity of IL4I1 (such as inhibitory molecules specific for IL4I1) or an antagonist that alters expression or activity of at least one downstream target of IL4I1. Thus, also provided are uses for one or more antagonists that reduce or inhibit IL4I1 activity in the manufacture of a medicament for the treatment or prevention of a respiratory disease. Also provided are antagonists that reduce or inhibit IL4I1 activity for use in the treatment or prevention of a respiratory disease. Also provided are pharmaceutical compositions for use in the treatment or prevention of a respiratory disease, wherein the composition includes one or more antagonists that reduce or inhibit IL4I1 activity, together with at least one pharmaceutical carrier, diluent or excipient.

In some examples, such methods and compositions decrease IL4I1 activity by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such as an amount of IL4I1 activity in the absence of treatment with an antagonist of IL4I1 or prior to treatment with an antagonist of IL4I1). In some embodiments, the disclosed methods include measuring the activity of IL4I1, such as monitoring the enzymatic activity of IL4I1 by measuring levels of particular cleavage products. In another embodiment, the disclosed methods include measuring the activity of IL4I1 by monitoring the effect of IL4I1 on immune cell responses by gene expression changes, protein expression or functional responses.

In some examples, such methods and compositions decrease expression of IL4I1 (such as expression of a IL4I1 nucleic acid or protein) by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such as an amount of expression in the absence of treatment with an IL4I1 antagonist or prior to treatment with an IL4I1 antagonist). In some embodiments, the disclosed methods include measuring the nucleic acid or protein expression of IL4I1.

In one example, the expression and/or activity of IL4I1 is decreased by using the disclosed compositions and methods. The disclosed inhibitors/antagonists can be specific for IL4I1 gene sequences, coding sequences, and protein sequences. Exemplary sequences that can be targeted with the disclosed methods and compositions are known (for example from the GenBank® database of nucleic acid and protein sequences, examples of which are provided herein). In addition, one skilled in the art will appreciate that variant sequences which retain IL4I1 activity can be targeted. For example, such variants may include encode a protein with one or more deletions, substitutions, or additions (or combinations thereof), such as 1-50 of such changes (such as 1-40, 1-30, 1-20, or 1-10 of such changes). In certain examples, an IL4I1 nucleic acid sequence or protein sequence targeted by the disclosed methods or compositions has at least 80%, at least 85%, at least 90%, at least 95%, at least 97%, at least 98% or at least 99% sequence identity to a known IL4I1 sequence.

Exemplary respiratory diseases that can be treated or prevented with the disclosed methods and compositions include, but are not limited to: COPD, exacerbated COPD, asthma, bronchitis, emphysema, cystic fibrosis, acute respiratory distress syndrome, bronchiectasis, or combinations thereof. In some examples, the COPD exacerbation results from a respiratory infection, such as a bacterial infection (e.g., infection by one or more of Moraxella catarrhalis, Haemophilus influenzae, Streptococcus pneumoniae, and/or mycobacteria such as Mycobacterium tuberculosis) or a viral infection (e.g., infection by HRV or RSV). In some examples, the respiratory infection is a secondary bacterial infection. Thus, subject can have COPD, exacerbated COPD, asthma, bronchitis, emphysema, cystic fibrosis, acute respiratory distress syndrome, bronchiectasis, or combinations thereof. In some examples, the subject has a respiratory infection, such as a bacterial infection (e.g., infection by one or more of M. catarrhalis, H. influenzae, S. pneumoniae, and/or mycobacteria such as Mycobacterium tuberculosis) or a viral infection (e.g., infection by HRV or RSV).

Treatment of a respiratory disease, such as COPD or exacerbated COPD, by antagonizing or inhibiting IL4I1 activity (e.g., by decreasing the expression or activity of IL4I1) can include delaying the development of the respiratory disease in a subject (such as preventing development of exacerbated COPD). Treatment of a respiratory disease also includes reducing signs or symptoms associated with the respiratory disease (for example by reducing neutrophil count, reducing sputum production, reducing shortness of breath, reducing cough, improving pulmonary function, or combinations thereof). In some examples, reductions of at least 10%, at least 20%, at least 50%, at least 75%, at least 90%, or at least 95%, for one or more of neutrophil count, sputum production, shortness of breath, cough are achieved by the disclosed compositions and methods. In some examples, pulmonary function is improved by at least 10%, at least 20%, at least 50%, at least 75%, at least 90%, at least 95%, at least 100%, at least 200% or at least 500%, by the disclosed compositions and methods.

The subject can be any mammalian subject, including human subjects, non-human primates, laboratory mammals, and veterinary subjects such as horses, cats and dogs. The subject can be a child or an adult. In some examples, the subject is a smoker. In some examples, the method includes selecting a subject with a respiratory disease, such as COPD, exacerbated COPD, asthma, bronchitis, emphysema, cystic fibrosis, acute respiratory distress syndrome, bronchiectasis, or combinations thereof. These subjects can be selected for treatment with one or more antagonists that decrease IL4I1 activity, for example by decreasing IL4I1 gene expression, protein expression, and/or biological activity.

The disclosed methods include use of one or more antagonists that inhibit IL4I1 activity. Such antagonists are administered to subjects in therapeutically effective amounts induce the desired response (e.g., treatment or prevention of a respiratory disease). In some embodiments, the antagonist of IL4I1 is a specific binding antagonist, such as an antibody or fragment thereof, functional nucleic acid (such as an aptamer), antisense molecule (or other inhibitory nucleic acid molecule, such as siRNAs, miRNAs, shRNAs and ribozymes), inhibitory protein, peptide mimetic, inhibitory ligand, or small molecule inhibitor, or the like. Such antagonists can bind with higher affinity to a molecule of interest (such as IL4I1), than to other molecules. In one example, the antagonist inhibits IL4I1, is one identified using the methods provided herein.

In one example, the antagonist of IL4I1 decreases expression of IL4I1. In some examples, combinations of IL4I1 antagonists are used. Such antagonists can alter the expression of nucleic acid sequences (such as DNA, cDNA, or mRNAs) and/or proteins. In some examples, inhibitory nucleic acid molecules specific for IL4I1 are used, such as an antisense nucleic acid molecule, small interfering RNA (siRNA), short hairpin RNA (shRNA), micro RNA (miRNA), ribozyme, or combination thereof.

In other examples, the antagonist of IL4I1 decreases the biological activity of IL4I1. In some examples, combinations of IL4I1 antagonists are used. In some examples, an antibody, antibody fragment, antibody conjugate, small organic molecule, small inorganic molecule, functional nucleic acid molecule (such as an aptamer), combination thereof, specific for IL4I1 is used.

The one or more antagonists that inhibit IL4I1 can be administered to humans or other mammals (such as laboratory mammals, for example mice, rats, chimpanzees, apes, as well as pets, such as dogs and cats) by any means, including orally, intravenously, intramuscularly, intraperitoneally, intranasally, intradermally, intrathecally, subcutaneously, via inhalation or via suppository. In one non-limiting example, the composition is administered via injection. In some examples, site-specific administration of the composition can be used, for example by administering one or more antagonists that antagonize or inhibit IL4I1 to lung tissue (for example by using an inhaler).

Reducing IL4I1 Biological Activity with Antagonists

In one example, IL4I1 biological activity is reduced or inhibited by use of an antagonist of IL4I1. For example, antagonists can be used to reduce or inhibit the activity of IL4I1, for example by reducing the catalytic activity of IL4I1 and/or preventing signaling from IL4I1 and/or downstream targets. Such reductions are desirable when upregulation of the protein (or increased protein activity) causes or results in disease.

Examples of antagonists that can be used to reduce or inhibit the biological activity and/or expression of IL4I1 include but are not limited to: antibodies (such as polyclonal antibodies, monoclonal antibodies, chimeric antibodies, camelid antibodies, or humanized antibodies), antibody fragments, aptamers, therapeutic proteins, domain antibody or dAb™, or any other specific binding antagonist with specificity for IL4I1 which reduces or inhibits activity and/or expression of IL4I1, for example by at least 50%, at least 60%, at least 75%, or at least 90%. The antagonist need not inhibit IL4I1 activity by 100%. In some examples, the antagonist reduces the biological activity of IL4I1 by at least 20%, at least 30%, at least 40%, at least 50%, at least 60%, at least 70%, at least 75%, at least 80%, at least 90%, at least 95%, at least 98%, or at least 99%, for example relative to such activity in the absence of the antagonist.

In one example, antagonists can be used to decrease or eliminate IL4I1 activity. In some examples, such methods decrease IL4I1 activity by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such as an amount prior to treatment). Exemplary IL4I1 antagonists target an IL4I1 sequence shown in GenBank® Accession No. CAI54291.1, CAI54292.1, AI54293.1, AAZ32711.1, AAI06601.1, AAM15530.2, NM_152899.1, NM_172374.2, NM_001258017.1, NM_001258018.1, NR_047577.1, DQ079589.1, AJ880386.1, or NM_010215.3 (or sequences having at least 80%, at least 85%, at least 90%, at least 92%, at least 95%, at least 98%, or at least 99% sequence identity to such sequences). Examples of antagonists that can be used to reduce IL4I1 activity include, but are not limited to, antibodies, antibody fragments, aptamers, small molecules, or any other specific binding antagonist that binds specifically to an IL4I1 protein and reduces its activity.

### Exemplary Therapeutic Responses

The preparations disclosed herein are administered in therapeutically effective amounts. An “effective amount” is amount of one or more antagonists sufficient to reduce or eliminate a symptom of a respiratory disease (such as COPD or exacerbated COPD), for example by reducing neutrophil count, reducing sputum production, reducing shortness of breath, reducing cough, improving pulmonary function, or combinations thereof. Thus, in some examples, the disclosed methods include measuring neutrophil count, sputum production, shortness of breath, cough, pulmonary function, or combinations thereof, for example over a period of time (such as before and after administration of the therapeutic antagonist(s)). In particular examples, a change in neutrophil count, sputum production, shortness of breath, cough, or pulmonary function, is determined relative to the neutrophil count, sputum production, shortness of breath, cough, or pulmonary function of the subject at an earlier time (for example, prior to treatment).

In one example, the methods and compositions provided herein, decreases respiratory disease progression, such as the rate of such progression (for example, decreases of at least 5%, at least 10%, at least 20%, or at least 50%, for example relative to no administration of, or prior to administration of, one or more antagonists that reduce or inhibit IL4I1 activity.

The following examples are provided to illustrate certain particular features and/or embodiments. These examples should not be construed to limit the disclosure to the particular features or embodiments described.

### Example 1

### Conditioning of Healthy moDC with COPD Bronchial Epithelial Cells (BEC) Drives an Altered Gene Expression Profile

To differentiate airway epithelium in vitro, healthy smoker derived bronchial epithelial cells (Lonza lot #7F3833, 489, 237440, 899; Epithelix lot #13086) and COPD patient derived bronchial epithelial cells (Lonza lot #3313, 498, 3207, 3379; Epithelix lot #AB63, 472) were expanded according to the Lonza protocol with the following modification: the cells were subjected to two passages in Lonza BEBM media with supplements as reported in Widdicombe et al. (BioTechniques 39:249-255, August 2005). Cells were seeded onto collagen coated 0.4 um 96 transwell plates at 15,000 cells per 75 μl of the above media, and 235 μl of the same media was added to the basolateral receiver plate. After 3 days (with a single apical media change of the same volume), the apical media was completely removed in an airlift. To induce differentiation the cells were then cultured for 18 days with PneumaCult-ALI Medium (StemCell Technologies, #05001).

DC were generated as follows. Monocytes were isolated from the buffy coats from 13 healthy volunteer blood donors by using CD14 positive selection on PBMCs (Miltenyi #130-050-201) on an AutoMACS Pro, resuspended in DC media (RPMI 1640, 10% heat-inactivated FBS, 55 μM (WE, non-essential amino acids) with 30 ng/ml GM-CSF and 20 ng/ml IL-4 (both R&D systems) at 10⁶ cells per ml in a flask, then incubated at 37° C. for 6 days. Monocyte and DC purity and phenotype were confirmed with surface marker flow cytometry. Mono-culture and co-culture of DC was performed as follows, with DC seeded at 50,000 cells per well in a 96 well plate. To initiate co-cultures, the transwell portion containing bronchial epithelial cells (BEC) was transferred to a receiver plate seeded with DCs. After 48 hrs of cell culture, DC were lysed and mRNA levels were measured by microarray. Samples were hybridized onto GeneChip® Human Genome U133 Plus 2.0 Arrays and analysis was performed in Array Studio v7.1. The disease effect of COPD BEC, as compared to healthy BEC, on DC expression of the selected genes (CXCL5, PTGES, S100A8, SERPINB2, THBS1), was considered to be statistically significant with a p value<0.05 and a fold change of >1.5, with individual data points representing independent healthy moDC donors.

### Example 2

### Treatment of moDCs in Co-Culture with Bronchial Epithelial Cells from COPD Patients with IL4I1 Enhances mRNA Expression of Dendritic Cell Genes

Co-cultures were initiated as described above. Human IL4I1 (R&D Systems #5684-AO) was serially diluted into the basolateral media of the airway epithelial cultures (235 μl BEBM mixed in a 1:1 ratio with DMEM). After 24 hr incubation, the transwell portion containing the BEC (COPD donor #AB63) was transferred to a receiver plate seeded with 50,000 DCs per well and the matching IL4I1 dose and media conditions. After 24 hr coculture the DCs were lysed and mRNA levels were measured with an Affymetrix Quantigene multiplex panel on a FlexMap 3D instrument. For each gene the relative luminescence was normalized to a housekeeper gene GAPDH.

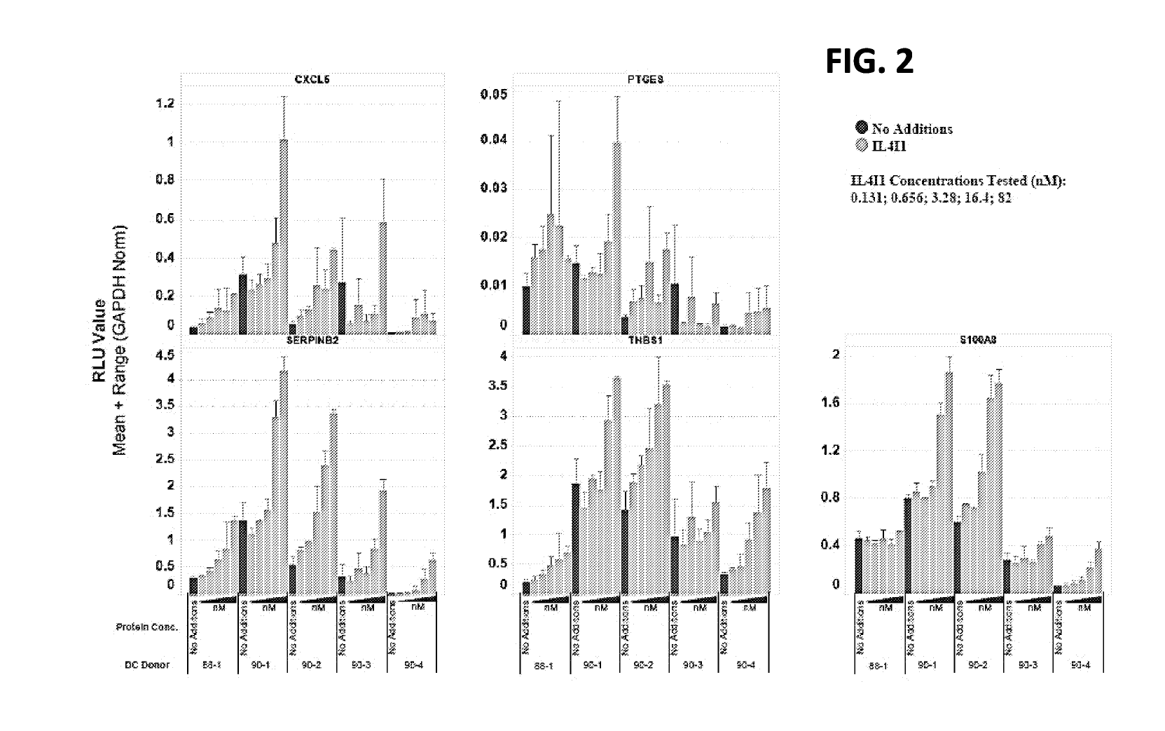

Epithelial cell interactions with DC influence DC maturation and subsequent innate and adaptive immune responses. In COPD, signals from epithelial cells may prime DCs to induce altered T cell responses and pro-inflammatory cascades. As shown in FIG. 2, treatment of moDCs in co-culture with epithelial cells from COPD patients with IL4I1 further enhances the expression of DC genes such as CXCL5, PTGES, S100A8, SERPINB2, THBS1, which are associated with a range of functions including neutrophil recruitment, pathogen recognition, immune tolerance, and T-cell responses. In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

1. A method of treating or preventing a respiratory disease or respiratory infection in a mammal, comprising reducing or inhibiting interleukin 4 inducible 1 (IL4I1) activity in the mammal.

2. Use of an antagonist that reduces or inhibits IL4I1 activity in the manufacture of a medicament for the treatment or prevention of a respiratory disease.

3. An antagonist that reduces or inhibits IL4I1 activity for use in the treatment or prevention of a respiratory disease.

4. A pharmaceutical composition for use in the treatment or prevention of a respiratory disease comprising one or more antagonists that reduce or inhibit IL4I1 activity, together with at least one pharmaceutical carrier, diluent or excipient.

5. The method of claim 1, wherein the method includes administering to the mammal a therapeutically effective amount of one or more antagonists that reduce or inhibit IL4I1 activity.

6. The method of claim 1, wherein the IL4I1 is a human IL4I1.

7. The method of claim 1, wherein the respiratory disease is one or more of chronic obstructive pulmonary disease (COPD), COPD exacerbation, asthma, bronchitis, emphysema, cystic fibrosis, acute respiratory distress syndrome and bronchiectasis.

8. The method of claim 7, wherein the COPD exacerbation results from a respiratory infection.

9. The method of claim 8, wherein the respiratory infection is a bacterial infection.

10. The method of claim 9, wherein the bacterial infection is an infection by one or more of Moraxella catarrhalis, Haemophilus influenzae, Streptococcus pneumoniae, and/or mycobacteria such as Mycobacterium tuberculosis.

11. The method of claim 1, wherein the respiratory infection is a viral infection.

12. The method of claim 11, wherein the viral infection is an infection by HRV or RSV.

13. The method of claim 8, wherein the respiratory infection is a secondary bacterial infection.

14. The method according to claim 1, wherein the IL4I1 activity is inhibited by an antagonist of one or more IL4I1 proteins.

15. The method of claim 14, wherein the antagonist of one or more IL4I1 proteins comprises an antibody, antibody fragment, antibody conjugate, aptamer, small molecules, therapeutic protein, domain antibody or dAb™, or combination thereof, specific for one or more IL4I1 proteins.

16. The method according to claim 1, wherein the mammal is a human.

17. The method according to claim 1, further comprising administering to the mammal one or more additional therapeutic antagonists to treat or prevent the respiratory disease.

18. A method of screening for an antagonist of IL4I1 activity for use in treating or preventing a respiratory disease comprising the step of determining whether the antagonist inhibits the IL4I1 pathway.
